(19) United States
(12) Patent Application Publication
(10) Pub. No.: US 2014/0113375 A1
(43) Pub. Date: Apr. 24, 2014

(54) Transient Expression And Reverse Transcription Aided Genome Alteration System

(71) Applicant: Lixin Liu, Beijing (CN)

(72) Inventor: Lixin Liu, Beijing (CN)

(21) Appl. No.: 14/058,214

(22) Filed: Oct. 19, 2013

Related U.S. Application Data
(60) Provisional application No. 61/716,592, filed on Oct. 21, 2012.

(51) Int. Cl. C12N 15/90

(52) U.S. Cl. CPC C12N 15/902
USPC 435/462; 435/320.1; 435/471

(57) ABSTRACT

Disclosed are methods and cellular systems for the generation of predetermined or random alterations at specific genomic location. The invention provides a transient expression and reverse transcription system to generate single-stranded DNA sequences homologous to a target genomic sequence, which can be transported to the nucleus to alter the genetic information of the target genomic sequence. Also provided are cellular and molecular components that can be used to increase the efficiency of the targeted genomic modification process.

### CROSS-REFERENCE TO RELATED APPLICATIONS

This application claims the benefit of U.S. provisional application No. 61/716,592, filed Oct. 21, 2012, the contents of which are incorporated by reference herein.

### BACKGROUND OF THE INVENTION

1. Field of the Invention

The present invention is in the field of molecular genetics. Particularly, it concerns using a transient expression and reverse transcription system to introduce targeted alterations in genomic sequences of host cells.

2. Description of the Related Art

Genetic modification techniques enable one to introduce changes to genetic information encoded in nucleic acid sequences of a living cell's and organism's genome. Conventional genetic modification techniques involve integration of a foreign nucleic acid sequence at a random location of a host genome. These methods are based on introduction of a vector containing a heterologous sequence into the host cell, random integration of the heterologous DNA into the host genome, and isolation of those cells with the heterologous sequence. These methods of illegitimate integration of genetic information into host genomes have several limitations. Due to random integration of the heterologous sequences, it may be integrated into and disrupt a functional gene essential for organism's survival. Even if the insertion of a heterologous gene does not compromise a host gene, the expression of the heterologous gene is affected by the surrounding genomic DNA (“positional effects”). In some cases, the negative effect of surrounding environment is so big that the heterologous gene cannot be expressed. In other cases, the genomic environment may cause overproduction of the heterologous gene which can have deleterious effect on the cell. Multiple copies of integrated heterologous genes can sometimes result in RNA interference. Another problem with these methods involves addition of unnecessary and unwanted genetic materials to the genome of the recipient, including, for example, viral or other vector remnants, control sequences, and marker genes. The addition of these unwanted genetic materials could have unexpected effects on the host organism over a long period of time, and selectable markers such as herbicide resistant genes and anti-antibiotics genes are major concerns for their negative impact on health and environment.

To solve the problems with conventional genetic modification techniques, targeted genetic modification technologies have been developed, which allows introduction of changes to specific locus of the host genome. These techniques are based on the site-specific correction or directed mutation of an episomal or chromosomal target sequence. Some of these techniques make use of different types of oligonucleotides or polynucleotides: double-stranded DNA (dsDNA), single-stranded DNA (ssDNA), oligonucleotides containing 5′ and/or 3′ modified ends to protect the molecule against cellular nuclease activities (Campbell et al., 1989), chimeric RNA/DNA or DNA/DNA molecules (Igoucheva et al., 2004a; Parekh-Olmedo et al., 2005), RNA oligonucleotides (Storici, 2008), and triplexforming oligonucleotides (Simon et al., 2008). Campbell et al. successfully used a single-stranded nucleotide to rescue a mutant neomycin phosphotransferase gene in the co-transfected plasmid. Zorin et al. used ssDNAs to insert a foreign DNA into a target position in the genome of the green alga Chlamydomonas reinhardti and reported that ssDNAs have much lower tendency than double-stranded DNAs (dsDNA) to undergo nonhomologous integration (Zorin et al., 2005). Kmeic et al. developed a method of using chimeric RNA/DNA duplex oligonucleotides to induce specific alterations in targeted genes of higher eukaryotic cells, and reported that the chimeric RNA/DNA duplex could give a higher rate of conversion than unmodified single-stranded oligonucleotides (U.S. Pat. No. 5,565,350). The molecular mechanism underlying the homology-based genetic modification is not fully understood. A possible mechanism involves the gene modification sequence hybridizes with the target sequence and creates mismatched bubbles that triggers DNA repair pathway, which uses the gene modification sequence to correct the target sequence in the host genome. Other possible mechanism involves strand invasion or conversion by way of homologous recombination.

One major drawback for the oligonucleotides based targeted modification techniques is the low conversion rate, which is partly due to the degradation and limited supply of the exogenous gene modification oligonucleotides/polynucleotides (Zorin et al., 2005). Kmeic et al. described a targeted modification technique using single-stranded oligonucleotide with nuclease-resistant modification such as phosphorothioate linkage, LNA linkage, and 2′-O-Me base, which could decrease cellular degradation of the oligonucleotides and increase the efficiency of conversion compared to unmodified single-stranded oligonucleotides or chimeric RNA-DNA duplex. With the optimized modifications, the conversion rate induced by modified single-stranded oligonucleotides can be 2-3 fold more than that induced by chimeric RNA/DNA duplex (U.S. Pat. No. 6,936,497). However, it is still poorly understood how the chemical modifications might interact with or influence proteins involved in the gene conversion process. Even an optimized oligonucleotide has been identified, in most cases, the successful conversion rate could still be relative low in the range of 1×10⁻⁴ (Zhu, 2000). Another problem with targeted gene modification is the competition from non-homologous integration of exogenous DNA. The ratio of non-homologous vs. homologous integration varies among different cell types and organisms. For example, yeasts have relatively high tendency of homologous recombination whereas non-homologous integration occurs at much higher frequency than homologous integration does in higher eukaryotic organisms like animals and plants. Zorin et al. found that the efficiency of homologous recombination is similar for ssDNA and dsDNA, whereas ssDNA has a much lower tendency than dsDNA to integrate at non-homologous locations (Zorin, 2005). This study provides a simple solution to reduce the non-homologous recombination.

Other gene targeting techniques involve using sequence targeting endonucleases such as transcriptional activator-like effector nucleases (Miller, et al., 2010), zing finger nucleases (Bibikova, Beumer, Trautman, & Carroll, 2003), or engineered homing endonucleases (Grizot, et al., 2009) in combination with gene modification vectors which contain sequence homologous to the gene to be targeted. The vector also includes a reporter gene and/or a selectable marker. These techniques are used to delete a gene, remove exons, add a gene, and introduce point mutations. The mechanism is believed to be homologous recombination. These techniques are fairly efficient, but they can only make one specific change at one time.

There is a need of technologies that can constantly provide a large quantity of gene modifying sequences in a relative long period of time to increase the efficiency of conversion in the targeted gene modification. The present invention satisfies such a need and provides other benefits such as introducing random mutations to a targeted genomic location as well.

### SUMMARY OF THE INVENTION

The present invention pertains to methods and cellular systems related to the generation of predetermined or random alterations at specific genomic location. The invention provides a transient expression system to generate a large and continuous supply of single-stranded DNA sequences homologous to a target genomic sequence, which can be transported to the nucleus to alter the genetic information of the target genomic sequence via DNA repair pathways or homologous recombination. Also provided are cellular and molecular components that can increase the efficiency of the targeted genomic modification process.

One embodiment of the invention is a system for introducing an alteration in a target genomic sequence of a host cell, comprising: a Genomic Sequence Modification Sequence (GSMS) expression cassette, which comprises a polynucleotide sequence homologous to the target genomic sequence and a primer binding sequence, and which can be transcribed to produce GSMS RNAs inside a cell; a reverse transcriptase expression cassette, which comprises a polynucleotide sequence encoding a reverse transcriptase; a means of co-introducing the GSMS expression cassette and the reverse transcriptase expression cassette into said host cells, whereby the GSMS RNAs are reverse transcribed to single stranded GSMS cDNAs (ssGSMS cDNA) by the reverse transcriptase (FIG. 7), and ssGSMS cDNAs are transported to the nucleus and direct an alteration in the target genomic sequence using cellular DNA repair or homologous recombination machinery. The target genomic sequence can be any genomic sequence of interest in the host cell, including, for example, exon sequences, intron sequences, un-transcribed regulatory sequences, direct or invert repeat sequences, and sequences within recombination hotspots.

In some embodiment, the GSMS and reverse transcriptase can be linked together and integrated into a single expression cassette in which the reverse transcriptase coding sequence locates near 5′ end is separated from GSMS by a sequence encoding a RNA that can form a hairpin secondary structure that terminates reverse transcription (FIG. 12).

In some embodiment, the GSMS comprises a sequence fully complementary to the target genomic sequence of the host cell, except for one or more nucleotide differences at pre-selected positions. The nucleotide difference can be a mismatch, a deletion, or an insertion. In some embodiment, GSMS comprises a heterologous polynucleotide sequence inserted between a homologous sequence complementary to the target genomic sequence. In some embodiment, the heterologous polynucleotide encodes a protein, which, when integrated into host genome, can serve as selection marker for selecting cells with the genome alteration. In some embodiment, the GSMS comprising a sequence fully complementary to the target genomic sequence and a reverse transcriptase with poor proofreading ability are co-introduced into host cells. Due to the poor proofreading ability of the reverse transcriptase, random nucleotide mutations are created in the cDNA of GSMS during the reverse transcription. These ssGSMS cDNAs with random mutations can be integrated into the target genomic sequence via DNA repair or homologous recombination system, and result in a library of cells with random mutations in the target genomic sequence, which can be used for screening of mutants of the target sequence with more desirable properties. In some embodiment, the 5′ end of said GSMS RNA is designed to form a secondary structure that can terminate the reverse transcription when the reverse transcriptase meets the secondary structure (FIG. 9).

The GSMS has a primer binding site for binding with a primer to initiate a reverse transcription reaction. In some embodiment, the primer binding site is a sequence complementary to 3′ end of a natural tRNA or an artificially made tRNA. The GSMS RNA can then use the natural tRNA or co-expressed artificial tRNA as the primer to initiate a reverse transcription reaction (FIG. 9). In some embodiment, the 3′ end of GSMS RNA is designed to includes a poly(U) tails. A poly(A) tail is added to the 3′ end of the GSMS RNA at the end of transcription in eukaryotic cells. The GSMS RNA with a poly(U)-poly(A) tail can self-anneal to provide a primer for reverse transcription (FIG. 8).

In some embodiment, the reverse transcriptase is a naturally occurring enzyme with RNA-dependent DNA polymerase activity, such as HIV reverse transcriptase, M-MLV reverse transcriptase, and AMV reverse transcriptase. The naturally occurring reverse transcriptase usually has little or no proof-reading activity, which can be used to produce mutations in ssGSMS cDNAs when such mutations are desirable. When mutations in ssGSMS cDNAs are not desirable, engineered reverse transcriptase with good proof-reading ability or naturally occurring reverse transcriptase with good proof-reading ability should be used instead.

In some embodiment, a single-stranded DNA (ssDNA) binding protein cassette is included to produce a ssDNA binding protein that can bind to ssGSMS cDNA, protect it from degradation, and facilitate the transport of ssGSMS cDNA to nucleus. Preferably, the ssDNA binding proteins are important players involved in homologous recombination or DNA repair. Examples of such ssDNA binding protein include replication protein A, RecA, Rad51, DMC1, ICP8, SSB, or any proteins/peptides with similar function. E. coli. RecA protein and its homolog proteins like Rad51 and DMC1 are especially preferable as they bind to ssDNA to form nucleoprotein filament and mediate critical steps in homologous recombination including homology search, DNA strand invasion, and homologous sequence pairing (Holthausen, 2010) (FIG. 3).

In some embodiment, the system includes a sequence targeting endonuclease expression cassette encoding a sequence targeting endonuclease. The sequence targeting endonuclease targets the same region of the target genomic sequence that is homologous to the GSMS. The sequence targeting endonuclease is an engineered restriction enzyme that comprises a sequence recognition domain designed to recognize a pre-defined DNA sequence and a DNA cleavage endonuclease domain. The sequence recognition domain can be selected from the group including Zinc Finger sequence recognition domains, Transcriptional Activator-like effectors (TALE) sequence recognition domains, and meganuclease sequence recognition domains. The DNA cleavage endonuclease domain can have the nuclease activity to cut two strands of a double-stranded DNA (dsDNA) to create a double strand break, or to cut only on one strand of a dsDNA to create a nick on the dsDNA.

In some embodiment, the system includes a siRNA expression cassette, wherein said siRNA expression cassette produces a siRNA that induces the degradation of the RNA transcripts of the target genomic sequence while siRNA does not have sequence homology with the GSMS RNAs. The siRNA can also be chemically synthesized and is co-introduced into said host cells along with said GSMS and said reverse transcriptase expression cassette.

In some embodiment, the GSMS or reverse transcriptase expression cassette, ssDNA binding protein expression cassette, and sequence targeting endonuclease expression cassette can be in the form of RNA, which can be directly translated into proteins of interest or used as templates for making cDNA.

In some embodiment, the GSMS expression cassette, and one or more of the protein expression cassettes are integrated in the same vector in a tandem manner. In some embodiment, more than one protein coding sequence and GSMS can be integrated into a combination expression cassette. For example, a reverse transcriptase and a ssDNA binding protein coding sequences can be linearly linked to GSMS under the control of a single promoter. The protein coding sequences locate near 5′ end of the construct and GSMS locates near 3′ end of the construct. A translational skipping sequence is inserted between two protein coding sequences and a sequence encoding a hairpin forming RNA is inserted at 5′ end of GSMS to terminate reverse transcription (FIG. 12),

In some embodiment, it is desirable to select cells with the genome alteration. The cells with the altered genome can be selected by conventional methods. For example, the altered genome sequence may confer unique or beneficial property such as growth advantage, drug resistance, alternate metabolite usage, and fluorescence emission, which can be used as a base for selection. Alternatively, PCR and DNA sequencing of the target genomic sequence can be used to search for cell clones having expected mutations.

Another embodiment of the present invention provides a method of introducing an alteration in a target genomic sequence of a host cell, comprising the steps of: a. constructing a GSMS expression cassette that comprises a polynucleotide sequence homologous to the target genomic sequence and a primer binding sequence, and that the GSMS expression cassette produces GSMS RNAs; b. constructing a reverse transcriptase expression cassette encoding a reverse transcriptase; c. co-introducing the GSMS expression cassette and the reverse transcriptase expression cassette into the host cells, whereby the GSMS RNAs are reverse transcribed to ssGSMS cDNAs by the reverse transcriptase, and the ssGSMS cDNAs direct the alteration in the target genomic sequence via DNA repair pathways and/or homologous recombination. Optionally, the method further includes constructing one of more expression cassettes selected from the group including a primer expression cassette, a ssDNA binding protein expression cassette, a sequence targeting endonuclease expression cassette, and a siRNA expression cassette.

Another embodiment of the present invention provides a method of obtaining a population of cells with random mutations in a target genomic sequence, comprising the steps of: a. constructing a GSMS expression cassette that comprises a polynucleotide sequence fully complementary to the target genomic sequence and a primer binding sequence; b. constructing a reverse transcriptase expression cassette encoding a reverse transcriptase with poor proofreading ability; c. co-introducing the GSMS expression cassette and the reverse transcriptase expression cassette into host cells, whereby said GSMS RNAs are reverse transcribed to ssGSMS cDNAs with random mutations by the reverse transcriptase with poor proofreading ability, and the ssGSMS cDNAs direct the integration of random mutations into said target genomic sequence; d. collecting cells with random mutations in the target genomic sequence. Optionally, the method further includes constructing one of more expression cassettes selected from the group including a primer expression cassette, a ssDNA binding protein expression cassette, a sequence targeting endonuclease expression cassette, and a siRNA expression cassette.

### BRIEF DESCRIPTION OF THE DRAWINGS

FIG. 1. The basic GSMS transient expression system. The plasmid includes a GSMS expression cassette, a reverse transcriptase expression cassette, and a ssDNA binding protein expression cassette, each having its own promoter and terminator.

FIG. 2. The GSMS transient expression system with sequence targeting endonuclease. The plasmid includes a GSMS expression cassette, a reverse transcription expression cassette, a ssDNA binding protein expression cassette, a tRNA expression cassette, and a sequence targeting endonuclease expression cassette, each having its own promoter and terminator.

FIG. 3. The scheme showing production of ssGSMS cDNAs inside cells. The GSMS is first transcribed into RNA and converted to single stranded cDNA with the help of co-expressed tRNA as primer and reverse transcriptase. The ssGSMS cDNA is then associated to ssDNA binding protein and enters the nucleus with or without sequence targeting endonuclease.

FIG. 4. Possible application of ssGSMS cDNA after it enters nucleus when sequence targeting endonuclease is not included. The ssDNA binding protein not only protects ssGSMS cDNA from degradation and formation of secondary structure but also help search and align ssGSMS cDNA to the homologous genomic region. During alignment, the single nucleotide mutation on ssGSMS cDNA either by design or through error prone reverse transcriptase form mismatch bubbles which triggers host cell's mismatch repair (MMR) pathways and introduce mutations into genomic DNA. It is also possible that ssGSMS cDNA gets integrated into host cell's genome through homologous recombination or similar processes, e.g. strand invasion and strand exchange.

FIG. 5. Possible application of ssGSMS cDNA after it enters nucleus when sequence targeting double strand cutting endonuclease is included. The ssDNA binding protein not only protects ssGSMS cDNA from degradation and formation of secondary structure but also help search and align ssGSMS cDNA to the homologous genomic region. The sequence targeting endonuclease creates a double strand break (DSB) at the target locus of the genome and makes the target locus available for ssGSMS cDNA to hybridize to the homologous region. After alignment, the ssGSMS cDNA may serve as a template to direct the synthesis of the target genomic sequence or be integrated into the target genomic sequence by way of homologous strand crossover.

FIG. 6. Possible application of ssGSMS cDNA after it enters nucleus when sequence targeting single strand nicking endonuclease is included. The ssDNA binding protein not only protects ssGSMS cDNA from degradation and formation of secondary structure but also help search and align ssGSMS cDNA to the homologous genomic region. The sequence targeting endonuclease creates a single strand nick at the target locus of the genome and makes the target locus available for ssGSMS cDNA to hybridize to the homologous region. After alignment, the ssGSMS cDNA may serve as a template to direct the synthesis of the target genomic sequence or be integrated into the target genomic sequence by way of homologous strand crossover. Using sequence targeting nicking endonuclease is less likely to cause off-site mutation or chromosome rearrangement than using sequence targeting double strand cutting nuclease.

FIG. 7 shows production of ssGSMS cDNA using tRNA as primer. A primer binding site complementary to 3′ end of a tRNA is designed at 3′ end of GSMS RNA. When GSMS is transcribed into RNA, tRNA binds to the primer binding site of GSMS RNA and serves as a primer to initiate the reverse transcription reaction.

FIG. 8 shows poly(T/A) priming for ssGSMS cDNA synthesis. It is designed that a poly(dT) sequence is added to the 3′ end of GSMS (+) strand so that a poly(U) sequence will be included in the 3′ end of GSMS RNA. During the transcription, a poly(A) tail is added to the 3′ end GSMS RNA following the poly(U) tail. The poly(A) tail can fold back to anneal to the poly(U) tail and serve as a primer for the reverse transcription synthesis of ssGSMS cDNA.

FIG. 9 shows a GSMS RNA with a 5′ secondary structure that terminates reverse transcription. GSMS is designed so that the 5′ end of GSMS RNA forms a hairpin secondary structure. When the reverse transcriptase encounters the secondary structure at 5′ end of GSMS RNA, it terminates the reverse transcription reaction.

FIG. 10 Shows the action of reverse transcriptase with good or poor proofreading activity. When reverse transcriptase with good proofreading activity is used, GSMS is converted into ssGSMS cDNA as it is designed. When reverse transcriptase with poor proofreading activity is used, random mutations are likely introduced into ssGSMS cDNA by the enzyme and result in a library of ssGSMS cDNA with random mutations inside host cell.

FIG. 11 shows some of expression cassettes that can be included in the system to facilitate the targeted genome modification.
Cassette 1: promoter/GSMS/terminator
Cassette 2: promoter/siRNA of GSMS targeting gene/terminator
Cassette 3: promoter/reverse transcriptase/terminator
Cassette 4: promoter/artificial tRNA that binds to primer binding site within GSMS/terminator
Cassette 5: promoter/natural tRNA that binds to primer binding site within GSMS/terminator
Cassette 6: a fluorescent protein cassette
Cassette 7: other RNAi cassettes to interfere with RNA transportation, RNA degradation.
Cassette 8: promoter/Zinc Finger nuclease or TALEN/terminator
Cassette 9: promoter/ssDNA binding proteins/terminator

FIG. 12 Shows the scheme of a combination expression cassette in which more than one protein coding sequences (e.g. reverse transcriptase and ssDNA binding protein coding sequences) are linearly linked to GSMS and expressed from the same promoter. The protein coding sequences locate near 5′ end of the cassette and GSMS near 3′ end of the construct. A translational skipping sequence is inserted between two neighboring protein coding sequences and a sequence encoding a RNA with a hairpin structure is inserted at the 5′ end of GSMS. After the cassette is transcribed into a RNA, ribosomes start protein translation from 5′ end of the RNA and terminate at the stop codon, whereas reverse transcriptases start making cDNA from 3′ end of the RNA and stop at the hairpin secondary structure without stepping into the protein coding region. The translational skipping sequence encodes a self-cleaving peptide, allowing generation of a separate reverse transcriptase and a ssDNA binding protein instead of a large fusion protein.

### DETAILED DESCRIPTION

Targeted genome modification technologies rely on the provision of gene modification nucleic acid sequences homologous to specific target sequences inside host cells. A number of techniques make use of different types of oligonucleotides or polynucleotides, including dsDNA, chimeric DNA/RNA, modified ssDNA, single-stranded oligonucleotides, and triplex-forming oligonucleotides. Once introduced into the cell, these polynucleotides/oligonucleotides quickly start to be degraded by cellular nucleases. Unmodified oligonucleotides microinjected inside mammalian cells have a half life of 15 to 20 minutes, and modified oligonucleotides can have a longer half life of several hours depending on the type and position of the modification (Fisher, 1993) (Jean Paul Leonetti, 1991). Provision of a large and constant supply of gene modification sequences inside cells can greatly increase the efficiency of targeted gene alteration.

The present invention provides a transient expression system that can generate a large and continuous supply of single-stranded cDNA sequences with at least a portion of it homologous to a target genomic sequence, which can be transported to the nucleus and direct the alteration of the target genomic sequence. The present invention also provides a method of generating a library of cells with random mutations integrated in the targeted genomic sequence. Another aspect of the invention combines the usage of ssGSMS cDNA and sequence targeting endonucleases such as transcriptional activator-like effector nucleases (Miller, et al., 2010), zing finger nucleases (Bibikova, Beumer, Trautman, & Carroll, 2003), or engineered homing endonucleases (Grizot, et al., 2009) to introduce alterations in targeted sequence. Also provided are cellular components and molecular means that can increase the efficiency of the targeted genomic modification.

The core of the present invention is a transient expression system that mimics the replication system of the retroviruses or retro-transposons, which enables cellular amplification of a Genomic Sequence Modification Sequence firstly by transcription and secondly by reverse transcription. Because of the two rounds of amplification, it is possible to provide a continuous and large quantity of supply of ssGSMS cDNA, available for participating in the cellular process leading to the desired alteration of the target sequence. After several days, the transient expression system can be completely degraded inside the cells, leaving no trace of genetic materials of the system itself but the desired alteration in the target sequence. Compared to techniques using oligonucleotides, the present invention can provide a relatively constant and high levels of ssGSMS cDNAs over a period of several days, even several weeks, not a few hours. The present invention is especially useful for hard-to-transfect cells (e.g. plant cells), where small amount of expression vectors transferred into the cells can provide large amount of ssGSMS cDNA to participate in the homology-directed gene alteration. Furthermore, as dsDNA is 100 fold more likely than ssDNA to engage in non-homologous integration (Zorin et al, 2005), this invention using ssDNA to direct targeted gene alteration can greatly reduce illegitimate DNA integration compared to methods using dsDNA (e.g. gene modification plasmids).

Another aspect of the present invention makes use of the poor proofreading ability of reverse transcriptase. Using a reverse transcriptase having poor proofreading ability, ssGSMS cDNA with random mutations can be continuously generated inside host cells, which can direct integration of random mutations into the target gene via homology-based mechanisms. The library of cells with random mutations in the target gene can be used for screening of new traits and desirable phenotypes.

Also provided in the system of the invention are ssDNA binding proteins that can stabilize and facilitate nucleus transport of ssGSMS cDNA, and sequence targeting endonucleases that can make a single strand nick or a double strand break at the location where the specific alteration is to be made. The present invention also provide a method of using siRNA to keep the target sequence in the unwound state so that it is more accessible to DNA hybridization and enzyme manipulation. This present invention can be used to introduce alterations to a specific endogenous gene as well as to integrate a piece of heterologous DNA into a specific location of the episomal or chromasomal DNAs.

Unless otherwise defined, all the technical and scientific terms will be used in accordance with common understanding of persons ordinary skilled in the art to which the present invention is related. As used herein, the following terms shall have the assigned meanings unless a contradictory definition is clearly indicated from the context in which the term is used.

The term “genomic sequence modifying sequence (GSMS)”, as used herein, refers to a nucleic acid sequence that can be used to make alternations to a target sequence at a defined locus of host chromosomal or episomal DNA. The alternations can be one or more nucleotide mutations, insertions, deletions, or the combination of the above. The alteration can also be insertion of a heterologous DNA conferring a desired phenotype. The term “episomal DNA” refers to an extra-chromosomal DNA in the cells that can replicate independently, including, for example, DNAs for plasmids, cosmids, bacterial artificial chromosome (BAC), and yeast artificial chromosome (YAC). The GSMS comprises a primer binding sequence and a sequence homologous to a target sequence of host chromosomal or episomal DNA. The homologous sequence of the GSMS can be fully complementary to the target sequence, except for one or more nucleotide difference. The GSMS can also have various degree of homology to the target sequence, for example, with 10-20%, 30-40%, 50-60%, 70%, 80%, 90%, 95% sequence homology. In some embodiment, the GSMS comprises a heterologous sequence flanked at one side or both sides by a sequence homologous to the target sequence. The homologous sequence of the GSMS should be in sufficient length so that it can specifically recognize and hybridize to the target sequence in the host cell. The length of the homologous region can be, for example, at least 10 base pairs, or preferably at least 40-60 base pairs, or even more preferably a few hundreds or thousands of base pairs. A target sequence can be any sequence of interest that is subjected to be changed or modified, including coding sequences and noncoding sequences such as regulatory sequences, intron sequences, and repeat sequences. The target sequence can be, for example, a sequence residing in the recombination hotspot or a sequence with direct or inverted repeats that have a high tendency for homologous recombination.

The terms “exogenous DNA”, “heterologous polynucleotide”, “heterologous nucleic acid”, or “heterologous sequence”, as used herein, refer to one that originates from a source foreign to a particular host cell, or, if from the same source, it is in a locus in which the element is not ordinarily found in the host cell. Heterologous DNA sequence is expressed to yield a heterologous polypeptide, for example, a marker protein.

The terms “homologous sequence”, “homologous nucleic acid” or “homologous polynucleotide”, as used herein, refer to a nucleotide sequence (e.g. DNA or RNA) that originates from an endogenous source of a particular host cell. A homologous sequence can be isolated and cloned from the host cell or it can be chemically synthesized according to the blueprint of the endogenous nucleotide sequence. A homologous sequence can have identical or fully complementary sequence to an endogenous DNA or RNA sequence of the host cell. A homologous sequence may also contain certain nucleotide difference from the endogenous sequence, but it shall have high enough homology with the corresponding endogenous sequence so that, when introduced into the host cell, it can specifically recognize and preferably hybridize to the corresponding endogenous sequence. Homologous gene alteration refers to using a homologous nucleotide sequence to make changes to the corresponding endogenous gene in host cells.

The term “expression cassette” used herein, refers a part of nucleic acid sequence of a vector, which comprises genetic components needed to direct cellular machinery to make a RNA and/or a protein. The basic components of a DNA expression cassette include a promoter sequence, a sequence to make a RNA or a protein, and a transcription terminator sequence. A promoter is a region of DNA that binds to proteins (e.g. RNA polymerase, transcription factors) needed to initiate the transcription of a particular gene. Depending on the type of host cells (e.g. prokaryotic or eukaryotic cells), there are many promoter sequences commonly used in the design of expression cassettes. Promoters for prokaryotic cells include, for example, Lac promoter, Trp promoter, Tac promoter and T7 promoter. Strong viral promoters used in eukaryotic cell include, for example, cytomegalovirus immediate early (CMV-IE) promoter, simian virus 40 (SV40) promoter, Rous sarcoma virus long terminal repeat (RSV-LTR) promoter, and Moloney murine leukaemia virus (MoMLV) LTR promoter, and cauliflower mosaic virus (CaMV) 35S promoter. Eukaryotic promoters are generally weaker promoters than viral promoters, but they can have the advantage of tissue specific expression, for example, Apo A-I promoter (De Geest et al, 2000) and ApoE promoter (Kim et al, 2001) are liver specific promoters, and MCK promoter (Hauser et al., 2000) and myosin heavy-chain promoter (Skarli et al., 1998) are muscle specific promoters. A transcription terminator is a DNA sequence that causes RNA polymerase to terminate transcription. Prokaryotic and eukaryotic cells use different mechanisms to signal the end of a transcription, therefore having different termination sequences. The promoter sequence and the termination sequence in an expression cassette can be derived from the same gene or from different genes. For example, T7 termination sequence is used with T7 promoter sequence in expression cassettes for bacteria cells, and CMV-IE promoter can be used with a rabbit β globin terminator sequence in a mammalian expression cassette (e.g. pTandem-1 vector, Navagen, Madison, Wis.). Sometimes, it is possible to use a synthetic poly(A) termination sequence for this purpose (e.g. pTargeT™ vector, Promega, Madison, Wis.).

Besides using tandem gene expression cassettes to express multiple proteins, two or three proteins can be combined into a single expression unit driven by the same promoter, wherein DNA sequences encoding different proteins are separated by a translational skipping sequence such as the sequence encoding a self-cleaving 2A peptide (Halpin et al. 1999) (Zhang, 2013). 2A peptide is a self-cleaving picornavirus peptide with 18-22 amino acids. During protein translation, ribosomes skip the synthesis of the glycyl-prolyl peptide bond at the C-terminus of a 2A peptide, leading to the cleavage between the 2A peptide and its immediate downstream peptide (Kim, 2011). As showed in FIG. 12, one or more protein encoding sequences can be combined with GSMS sequence in one single expression cassette. The protein coding sequences are separated by a translational skipping sequence. GSMS is located close to the 3′ end of the expression cassette and is separated from the upstream protein coding sequence by a hairpin forming sequence. For example, when reverse transcriptase and ssDNA protein are combined with GSMS in an expression cassette, the expression of this cassette results in a RNA molecule with (from 5′ to 3′) a reverse transcriptase coding sequence, a translational skipping sequence, a ssDNA coding sequence, a hairpin forming sequence, followed by GSMS sequence (FIG. 12). The translational skipping sequence is translated into a self-cleaving peptide, allowing generation of a separate reverse transcriptase and a ssDNA binding protein. The stop codon at the end of ssDNA binding protein coding sequence prevents downstream GSMS RNA from being translated, and the hairpin structure at 5′ end of GSMS prevents reverse transcription from stepping into the protein coding region. The configuration allows multiple proteins or protein subunits being expressed from the same expression cassette, resulting in simpler expression constructs and more coordinated expression of related proteins.

The expression cassette is used to make a RNA or a functional protein. For example, a GSMS expression cassette is used to make a GSMS RNA, which can be reverse transcribed into a ssGSMS cDNA. A reverse transcriptase expression cassette, a ssDNA binding protein expression cassette, and a sequence targeting endonuclease expression cassette are used to express functional proteins. These protein expression cassettes are made to produce proteins or polypeptides having desired function without limitation to any particular protein/polypeptide sequences. The reverse transcriptase expression cassette is made to produce a naturally occurring or artificially engineered reverse transcriptase (RNA-dependent DNA polymerase) that can generate a complementary DNA from a RNA template. It may or may not possess RNase H activity, that is, catalyzing cleavage of RNA in a RNA-DNA duplex, and DNA-dependent DNA polymerase activity. The reverse transcriptase may be isolated from retroviral sources, for example, moloney murine leukemia virus (M-MuLV), human immunodeficiency virus (HIV) and avian myeloblastosis virus (AMV). The naturally occurring reverse transcriptases usually have little or no proof-reading ability, and is therefore prone to make errors during the reverse transcription. Engineered reverse transcriptase having good proof-reading activity is available from commercial sources like SuperScript™ Reverse Transcriptase (Life Technologies, Carlsbad, Calif.) and AccuScript High-Fidelity Reverse Transcriptase (Agilent Technologies, Santa Clara, Calif.). The high fidelity reverse transcriptase is needed when maintaining fixed mutation in a ssGSMS cDNA is desired. The reverse transcriptase with poor proof-reading ability can be used to make random mutations in ssGSMS cDNA.

The ssDNA binding protein expression cassette is made to produce a naturally occurring or artificially engineered ssDNA binding protein that can bind to a ssDNA, stabilize the ssDNA, and facilitate transportation of ssDNA into nucleus. Preferably, these ssDNA binding proteins are part of the cellular machinery for homologous recombination and can facilitate homologous recombination of GSMS. The ssDNA binding proteins can be, for example, replication protein A, RecA, Rad51, DMC1, ICP8, SSB and any homologous proteins having similar function. E. coli. RecA protein and its homologs are key proteins for homologous recombination that mediate homology recognition, homologous DNA pairing, and strand exchange. RecA and its eukaryotic homolog RAD51 can bind to ssDNA to form a dynamic nucleoprotein filament, perform homology search along dsDNA strand, and facilitate homologous DNA strand invasion (Li, 2008). Overexpression of RecA-like proteins may increase the efficiency of GSMS-directed homologous gene alteration. The sequence targeting endonuclease expression cassette is made to produce an endonuclease that can recognize a specific pre-defined nucleotide sequence. The sequence targeting endonuclease is an artificially engineered restriction enzyme that fuses a DNA recognition domain to a DNA cleavage domain. Examples of customizable DNA recognition domains include transcription activator-like effector (TALE) DNA binding domains (Wei, 2013) (Zhang, 2013) (Neville, 2013), Zinc Finger DNA binding domains (Bae, 2003), and meganuclease sequence recognition domains (Arnould, 2006) (Smith, 2006). The DNA recognition domain of the sequence targeting endonuclease can be designed so that the enzyme cutting site is close to the genomic locus to be changed. For example, an online application called TALEN Targeter (https://tale-nt.cac.cornell.edu/node/add/talen) is available for helping people to design a custom TALE nuclease (TALEN) for a specific target sequence. The DNA cleavage domain can be any catalytic domain for non-specific cleavage of DNA strand. A preferable example is the non-specific DNA cleavage domain of endonuclease Fok1. The DNA cleavage domain can be engineered so that the sequence targeting endonuclease is able to make either a double strand break or a single nick on one strand of a double-stranded DNA (Kim et al. 2012,).

In some embodiment, a siRNA expression cassette is used to generate a siRNA (small interference RNA) that can induce the degradation of the RNA transcript of the target sequence. The siRNA is designed to recognize a sequence outside of the homologous region of GSMS so that the siRNA will direct the specific degradation of the RNA transcript of the target sequence, but not the GSMS RNA. The degradation of the RNA of the target sequence may send a signal to the nucleus and keep the transcription of the target sequence in active state, thus maintaining the target sequence in the unwound or under-wound state. This creates a more accessible environment for GSMS to hybridize to the homologous region of the target site. There are many ways to make a siRNA expression cassette. For example, it can be made by using a RNA polymerase III promoter to drive the expression of a small hairpin siRNA and the transcription can be terminated by a stretch of unridines without the need of polyadenylation signal (Sui et al., 2002; Brummelkamp et al., 2002). siRNA expression Plasmid and viral vectors are available from commercial sources like Life Technologies, GeneScript, BMC Biotechnology.

The expression cassettes are preferably in the form of DNA. The expression cassette can also be in the form of RNA. A protein coding sequence in RNA expression cassette can be directly translated into proteins, and a GSMS RNA in a RNA expression cassette can be directly used as a RNA template to make a ssGSMS cDNA. For example, an integration deficient lentiviral vector (Chick, 2012) having a GSMS and a reverse transcriptase expression cassette can be used to generate ssGSMS cDNA. Integration-deficient retroviral vector is preferred as it reduces the possibility of illegitimate integration of the retroviral vector itself. A synthetic RNA molecule as showed in FIG. 12, which comprises multiple protein coding sequences under control of the same promoter and a GSMS sequence, can be introduced into host cells to directly generate ssGSMS cDNA and necessary proteins in a coordinated manner.

### The Production of Genomic Sequence Modifying Sequence

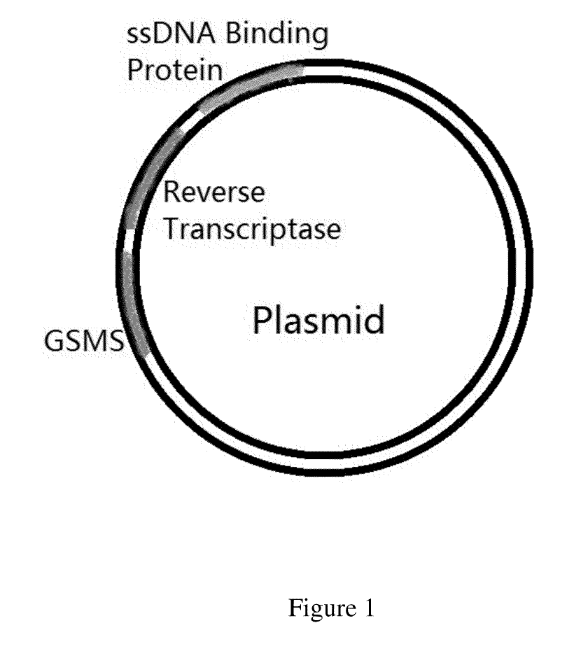

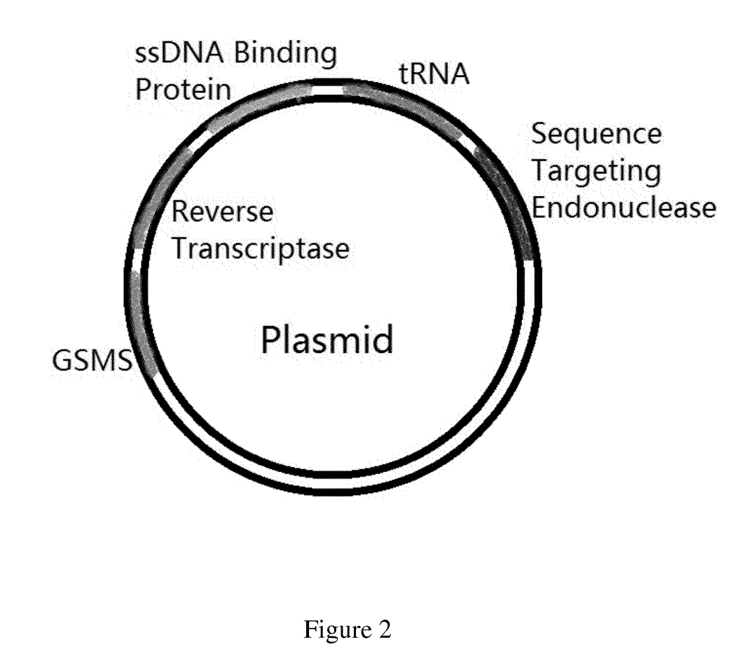

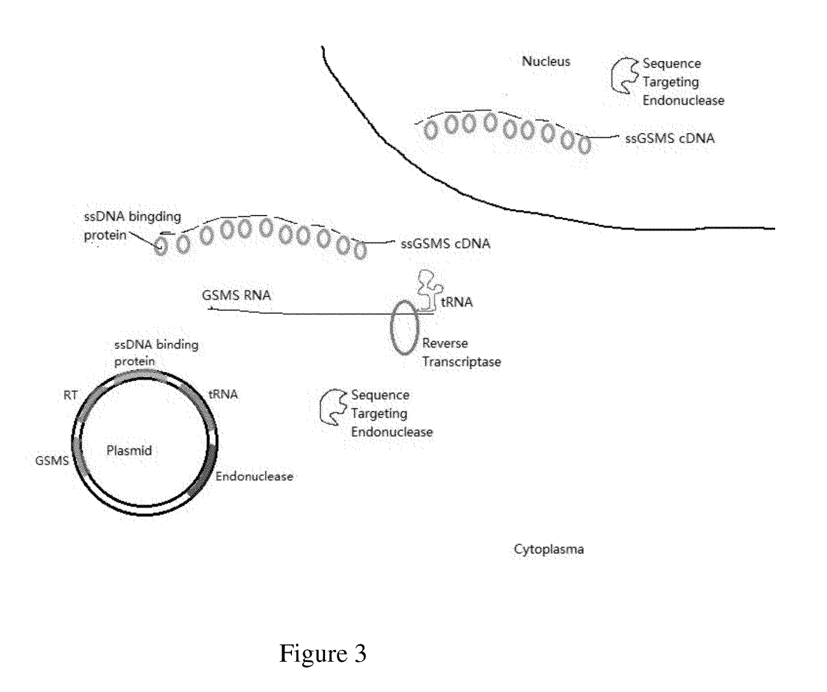

The GSMS is first cloned into an expression cassette with a promoter and terminator, and is introduced into host cells along with a reverse-transcriptase expression cassette residing in the same or a different vector (FIG. 1). Or GSMS and a reverse transcriptase can be combined into a single expression cassette as shown in FIG. 12. A ssDNA binding protein expression cassette (e.g. RecA, Rad51, RPA), either residing in the same or different vectors, can be co-transformed into host cells. Once inside the host cells, the GSMS is transcribed to RNA molecules. The GSMS RNAs are further reverse-transcribed to ssGSMS cDNA by expressed reverse-transcriptase using different priming methods (e.g. use tRNAs as primers). The ssGSMS cDNAs will then associate with expressed ssDNA binding protein to form nucleotides-protein complex or filament, which can protect ssGSMS cDNA from degradation, prevent the formation of secondary structure, facilitate the entry of ssGSMS cDNA into nucleus and increase alignment of ssGSMS cDNA with homologous genomic regions (Robyn L. Maher, 2013). For a few days or weeks while the vectors last, the GSMS can be amplified tens of thousands of times through transcription and reverse transcription.

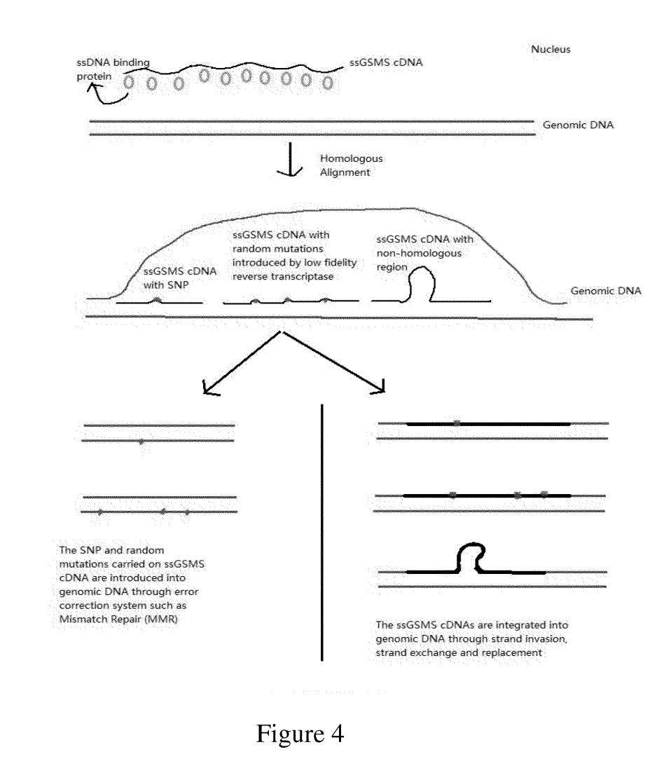

This presents a constant and large quantity of supply of ssGSMS cDNA sequences for entering host cell's nucleus, aligning with specific region of host cell's genomic DNA, and directing targeted gene alteration. The cellular mechanism of the homology-based gene alteration is yet to be fully understood. Possible mechanisms include different homologous recombination processes and DNA repair pathways. A possible DNA alteration mechanism through DNA mismatch repair (MMR) pathway is that the ssGSMS cDNA first hybridizes to the homologous region of the target sequence of host cell's genomic DNA. If ssGSMS cDNA contains difference(s) either through pre-design or introduced by error-prone reverse transcriptase, the alignment of ssGSM cDNA and the target sequence creates mismatched base pair(s). This will trigger host cell's gene repair or error correction systems, which may mistakenly use ssGSMS cDNA as template to correct the host cell's genomic sequence and therefore introduce mutation(s) into host cell's genome (FIG. 4). GSMS containing sequences homologous to targeted genomic sequence can participate in different homologous recombination processes, especially when target genomic sequence is damaged by sequence targeting endonuclease or other natural causes. The homologous recombination process generally involves homology search, DNA strand invasion, and template-directed DNA synthesis, resulting in gene alteration in a target sequence. The homologous recombination processes including but not limited to double-strand breaks repair pathway (DSBR), synthesis-dependent strand annealing pathway (SDSA), single-strand annealing pathway (SSA), break-induced replication pathway (BIR), RecBCD pathway and RecF pathway.

### GSMS Design for Different Application Purposes

If the system of the invention is to be used for changing predetermined base pairs of a host target gene, the homologous region of the GSMS should be fully complementary to the host target gene with the exception of the pre-determined nucleotide difference for base pair correction. The correcting nucleotide(s) are preferably located at or close to the center of the homologous sequence of the GSMS. A high fidelity reverse transcriptase should be used to reduce the probability of introducing unwanted mutation during the ssGSMS cDNA synthesis.

A desirable feature of the invention is that random mutations can be introduced to a target gene and a library of cells with random mutations in a target gene can be established. For this purpose, a GSMS with a sequence fully complementary to a target gene is used and a reverse transcriptase with poor proof-reading ability is used to convert GSMS RNAs into ssGSMS cDNAs with random mutations.

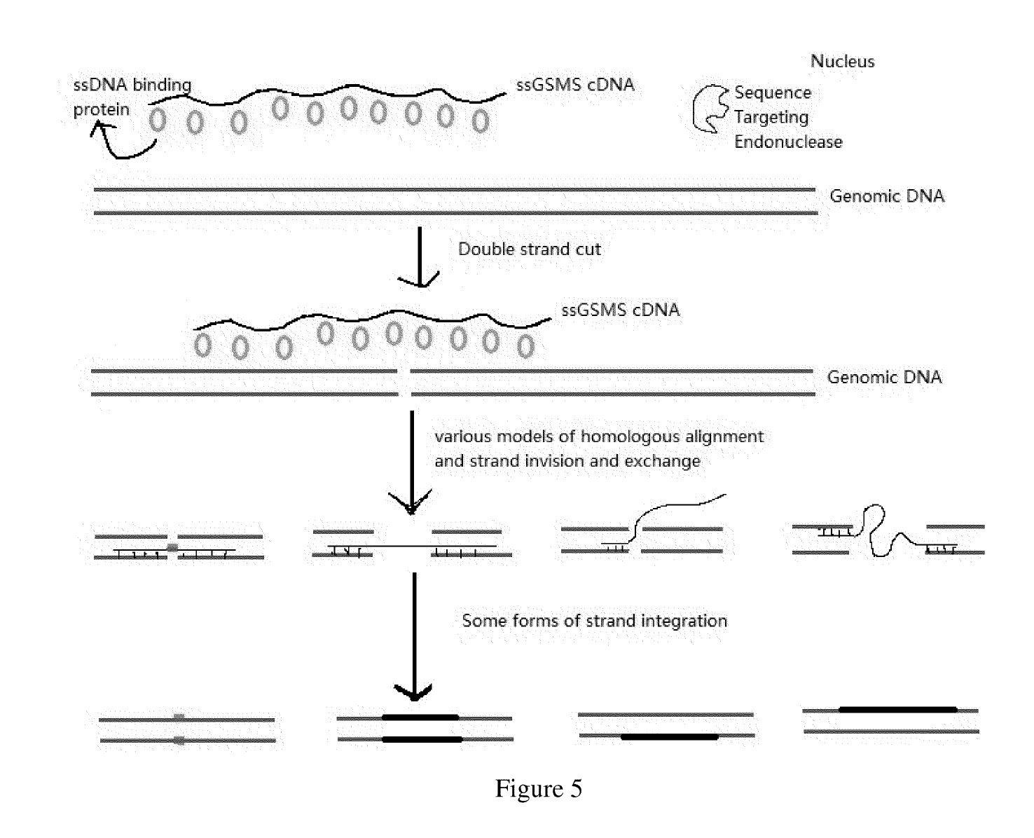

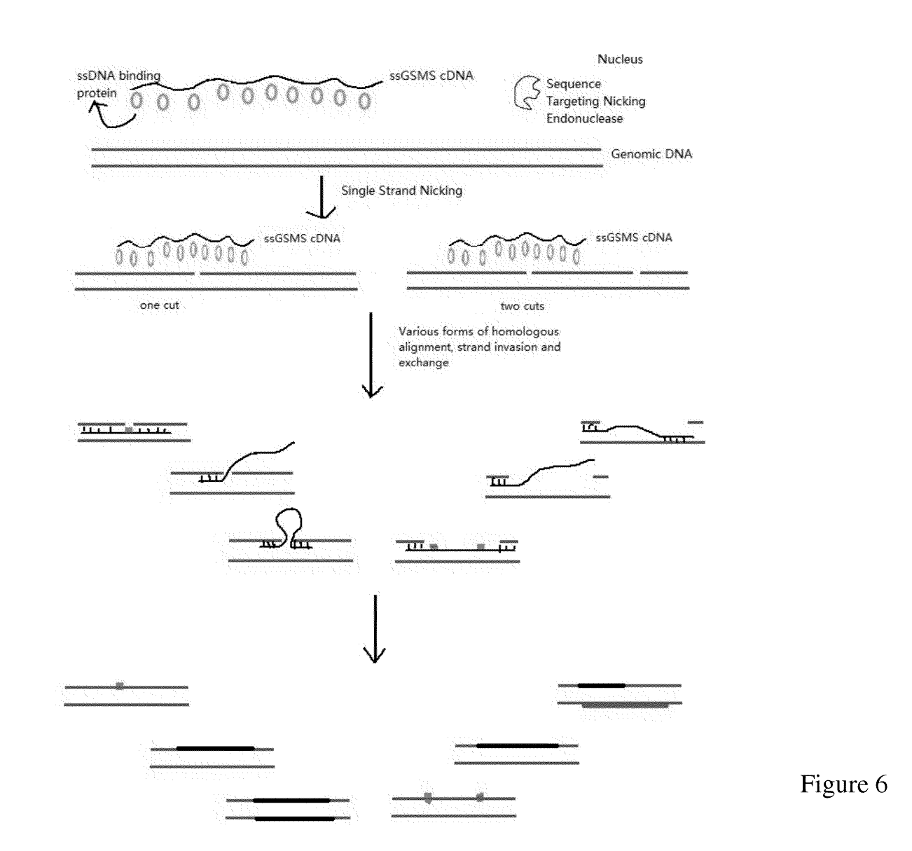

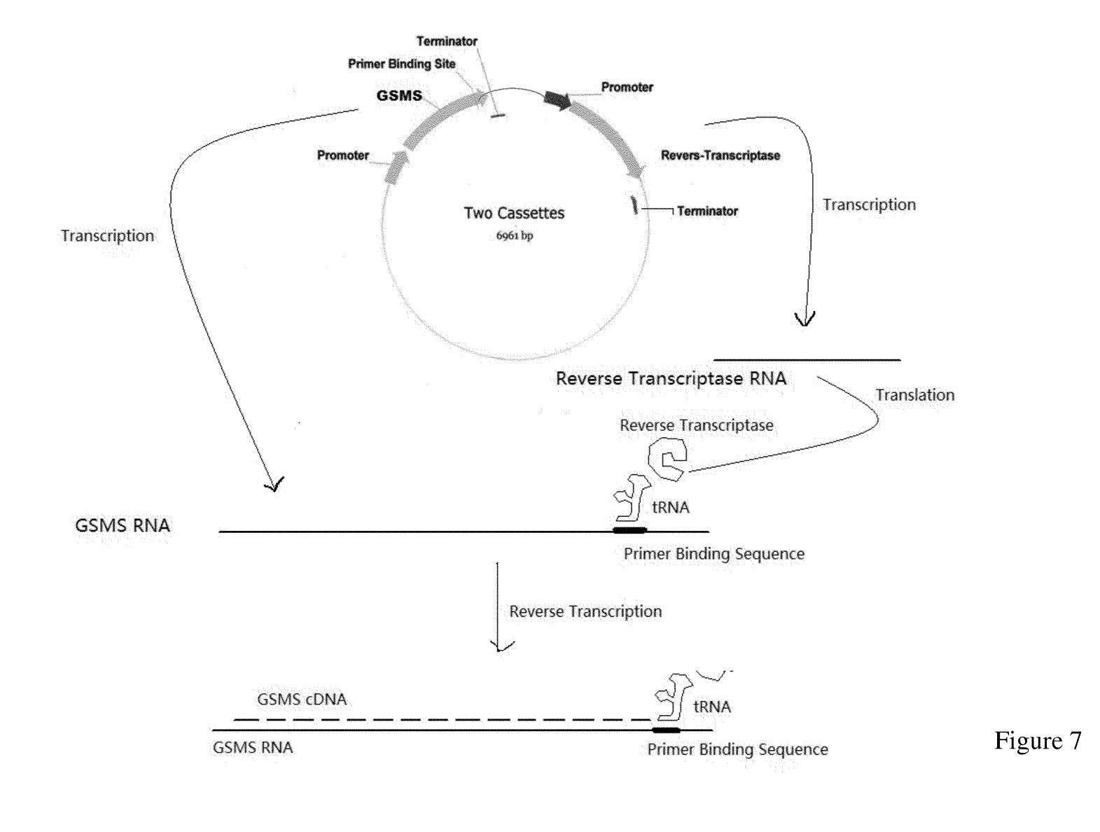

The system of the invention can also be used to integrate a heterologous DNA into targeted locus of host genome. For example, it can be used to knock-out an endogenous gene by inserting a junk DNA sequence or knock-in a desired gene at pre-defined genomic locus. For this purpose, an exogenous DNA with flanking homologous sequence at one side or both sides can be used in GSMS. The flanking homologous sequence is generally 100% complementary to the target sequence, and it should have sufficient length to support homologous recombination (FIGS. 5 and 6). The targeted location can be any genomic locus, for example, to increase the chance of targeted integration, the targeted sequence can be a sequence residing the recombination hotspot, or a sequence with direct or inverted repeats.

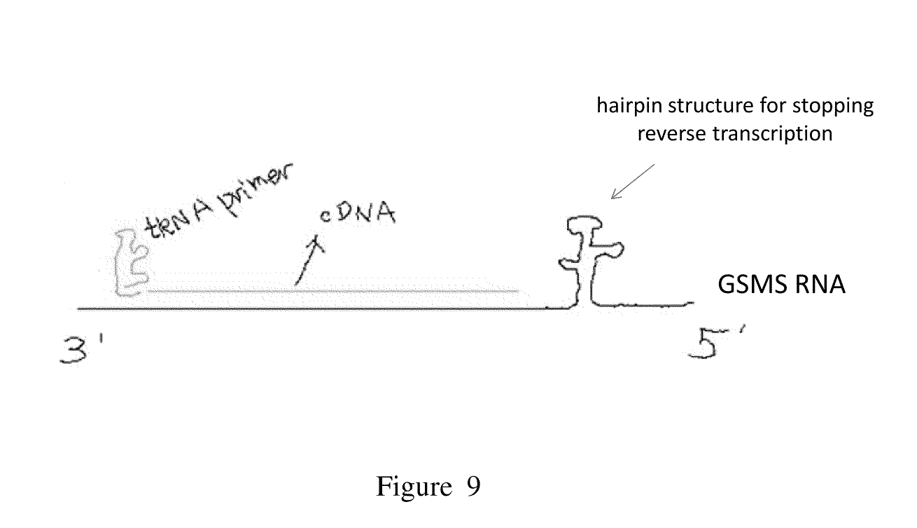

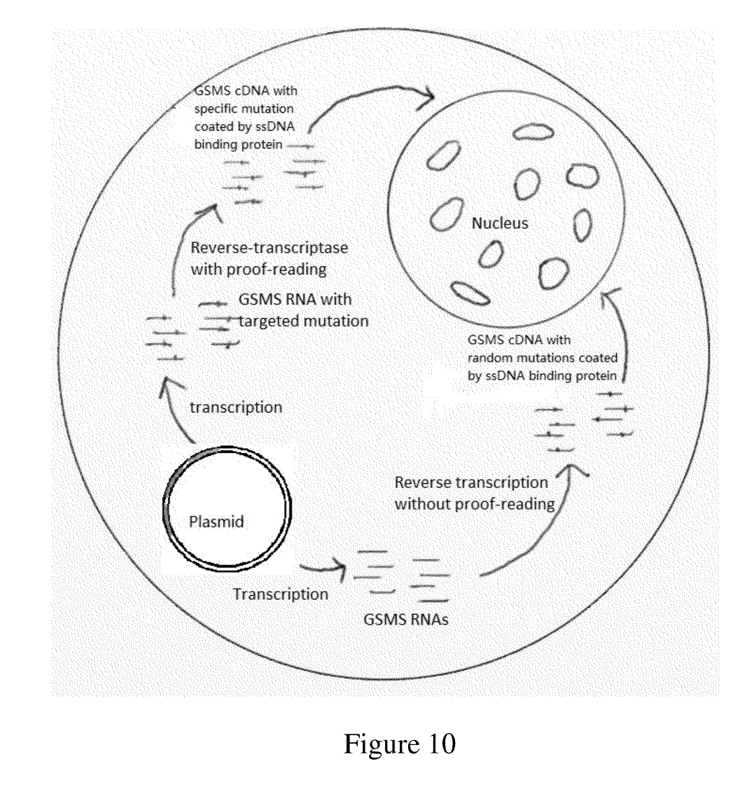

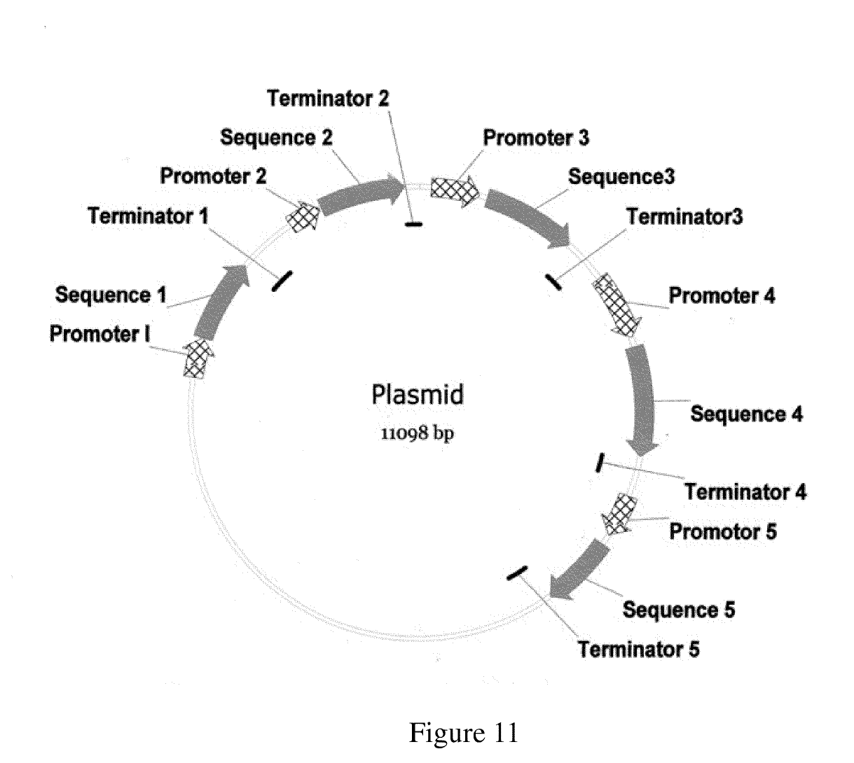

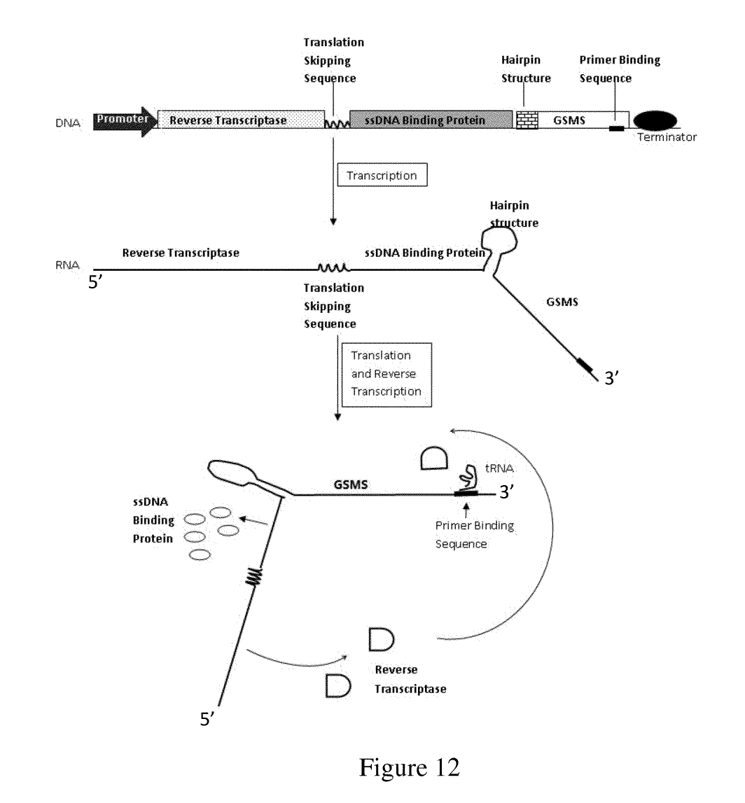

The GSMS DNA sequence can be designed in such a way that when transcribed into RNA, a hairpin secondary structure will form at the 5′ end of GSMS RNA. When reverse-transcriptase encounters the 5′ end secondary structure during the reverse-transcription, it falls off the RNA strand and terminates cDNA synthesis. This way no unwanted nucleotides will be added at 5′ end of ssGSMS cDNA (FIG. 9).

Priming Method for ssGSMS cDNA Synthesis

The preferable priming method for producing ssGSMS cDNA is to use natural tRNA or artificial tRNA as primers. Other priming methods like Poly(T/A) priming method can also be used.

tRNA priming method includes using natural tRNA or artificial tRNA for priming cDNA synthesis. 3′ ends of tRNAs have been used as primers for reverse-transcription by retro-viruses and retro-transposons (KLEIMAN, 1997) (R Marquet, 1995) (Voytas, 1993) (S. B. Sandmeyer, 1996) (Wakefield, 1995). Usually, 10-18 base pairs of 3′ end of tRNAs are used as primer to initiate reverse transcription in retro-viruses and retro-transposons (KLEIMAN, 1997). Reverse transcriptases from different retroviruses use different tRNAs as primers for initiation of reverse transcription. For example, reverse transcriptases from human immunodeficiency virus (HIV), Moloney Murine Leukemia Virus (M-MuLV), and Avian sarcoma leukosis virus (ASLV) use tRNA₃^{lys}, tRNA^{pro}, and tRNA^{trp}, respectively, as primer for initiation of cDNA synthesis (KLEIMAN, 1997). The primer binding sequence of GSMS can be designed to be fully complementary to the 3′ end sequence of the specific tRNA recognized by the reverse transcriptase of choice. GSMS RNAs can directly use host tRNA to initiate the reverse transcription reaction. A Natural tRNA expression cassette can also be added to the transient expression system along with GSMS and reverse-transcriptase cassettes to increase the available tRNA for priming and boost the ssGSMS cDNA production.

In addition to use host cell tRNA as a primer, artificial tRNA with exact match to a GSMS primer binding sequence can be designed as described in the reference (A H Lund, 1997). An artificial tRNA expression cassette along side with GSMS and reverse-transcriptase expression cassettes constructed in the same vector or different vectors is co-transferred to host cells. The artificial tRNAs serve as primers to initiate ssGSMS cDNA synthesis (FIG. 9).

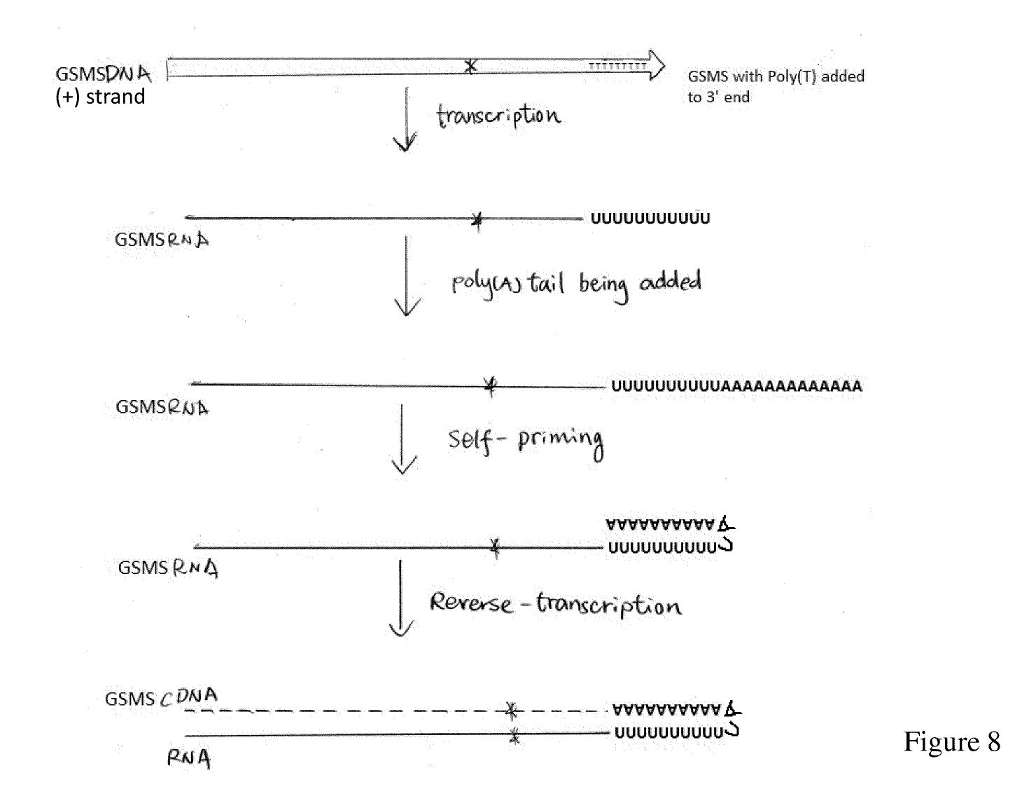

Poly(T/A) priming is to use Poly(A) tail added to the 3′ end of mRNA as a primer. To accomplish this, a poly(U) must be added to the 3′ end of GSMS RNA, correspondingly, a stretch of dT residues need to be added to the 3′ end of the sense strand of GSMS DNA. When a poly(A) tail is added after the poly(U) region at the 3′ end of GSMS RNAs, the poly(A) tail folds back to anneal to the poly(U) region and serve as primer for initiation of cDNA synthesis (FIG. 8). One potential problem is that the addition of poly(U) may compromise the transportation of RNA into cytoplasm. If that happens, a NLS (nucleus localization sequence) may be added to reverse-transcriptase and transport the enzyme into the nucleus where cDNA of GSMS can be synthesized.

### Selection of Reverse Transcriptase

For the system of the invention, it is desirable to select reverse transcriptase with RNase H, which can be used to release ssGSMS cDNA from RNA/cDNA hybrid molecules. For the purpose of precise correction of an error in a target sequence or insertion of a foreign gene into a target locus, the exact sequence of GSMS should be faithfully maintained during transcription and reverse transcription. Reverse-transcriptase with high proof-reading ability should be used to avoid introduction of unwanted mutations into ssGSMS cDNA during the reverse transcription. Since most naturally occurred reverse transcriptases lack 3′ to 5′ exonuclease activity (that is, proof-reading ability) and are therefore quite error prone, engineered reverse transcriptase with good proof-reading ability, for example, SuperScript® reverse transcriptase from Life Technologies, can be used for this purpose.

Reverse transcriptase with low proof-reading ability (Battula N, 1974) (Battula N, 1976) (Kunkel T A, 1981) can be used for a different purpose. For example, the error-prone HIV reverse transcriptase has an error rate of 1 in 1500 bases. For a 2 kb GSMS DNA, one random error will be created on average in one ssGSMS cDNA molecule. The error-prone reverse transcriptase can be used to generate random mutations in ssGSMS cDNA during reverse-transcription. The ssGSMS cDNA with random mutations can be used to direct integration of random mutations in the target gene, therefore creating a library of cells with random mutations in the target gene, which can be used to screen for the gene mutants with desirable properties.

Applications of ssGSMS cDNA

ssGSMS cDNA can be used to modify a target gene with or without help from sequence targeting endonucleases. In some embodiment, when a sequence targeting endonuclease is not included in the transient expression system, the ssGSMS cDNA may enter the nucleus, hybridize to the homologous region of host cell's genome. If ssGSMS cDNA contains difference(s) either through pre-design or introduced by error-prone reverse transcriptase, the alignment of ssGSM cDNA and homologous genomic region will create mismatched base pair(s). This will trigger host cell's gene repair or error correction systems like MMR and the gene repair or error correction system will mistakenly use ssGSMS cDNA as a template to correct the host cell's genomic sequence, therefore introducing mutation(s) into host cell's genome (FIG. 4). It is also possible that ssGSMS cDNA may hybridize to the homologous target sequence, and direct targeted alteration or integration of a foreign DNA in the host genome via homologous recombination processes.

In some embodiment, a sequence targeting endonuclease expression cassette is included in the transient expression system. A sequence targeting endonuclease is an artificially engineered nuclease that fuses a customizable DNA sequence recognition domain with a non-specific DNA cleavage domain. The sequence recognition domain can be customized to recognize a specific genomic region of 10 to 50 base pairs long. The sequence targeting endonuclease can be thus engineered to specifically recognize and make a DNA break at a genomic locus close to the targeted location where the alternation is expected. When large quantity of homologous ssGSMS cDNA is present in the nucleus, DNA breaks created at the target sequence activate homologous recombination pathways to repair the DNA damage. ssGSMS cDNAs form a nucleoprotein complex or filament with ssDNA binding proteins, perform homology search to find the DNA break at the homologous targeted sequence, probably with the help of ssDNA binding protein like RecA or homolog proteins, invade the homologous region and act as a template to direct DNA synthesis, therefore integrating the gene alteration at the targeted location. Combining ssGSMS cDNA and the sequence targeting endonucleases can greatly increase the efficiency of gene modification at the specific site via homologous recombination. The possible homologous recombination mechanisms involved include, but not limited to, double-strand breaks repair pathway (DSBR), synthesis-dependent strand annealing pathway (SDSA), single-strand annealing pathway (SSA), break-induced replication pathway (BIR), RecBCD pathway and RecF pathway.

Examples of sequence targeting endonucleases include TALEN, zinc finger nuclease (ZFN), and meganuclease. These sequence targeting endonucleases can be engineered to recognize and cut a specific dsDNA sequence of 10 to 50 base pairs long, making them great tools for targeted gene modification. TALEN is a preferable choice because the DNA recognition domain of TALEN comprises repeated highly reserved mini-motifs with strong correlation with individual nucleotides, making it possible to design a combination of TALEN mini-motifs to target any desired sequence (U.S. Pat. Nos. 8,440,431 and 8,440,432). The TALEN and ZFN can be further engineered to cut dsDNA to either create a double strand break (DSB) or to nick on only one strand of dsDNA (Kim, et al, 2012). Kim found that, when compared to DSB nucleases, nicking nucleases induce a lower integration rate. However, the frequency of off-site integration and chromosomal rearrangement is also much lower with nicking nuclease. If off-site integration and chromosomal rearrangement is a big concern, for example, in gene therapy, the sequence targeting nicking nuclease is a preferable choice. In some embodiment, peptides with affinity to each other can be fused to sequence targeting endonucleases (e.g. ZFN, TALEN) and ssDNA binding proteins, respectively. Through the interaction of the sequence targeting endonuclease and the ssDNA binding protein, ssGSMS cDNA associated with the ssDNA binding protein can then be brought towards cutting or nicking sites at the target location.

### Components of the Transient Expression System for Targeted Gene Alteration

The system described above has some basic components, additional expression cassettes can be added for various purposes (FIG. 11). The expression cassettes can be added to the system include, but not limited to:

Cassette 1: promoter/GSMS/terminator
Cassette 2: promoter/siRNA for degrading RNA transcript of the target gene/terminator
Cassette 3: promoter/reverse transcriptase/terminator
Cassette 4: promoter/artificial tRNA used as primer for reverse transcription)/terminator
Cassette 5: promoter/natural tRNA used as primers for reverse-transcription/terminator
Cassette 6: promoter/gene for fluorescent protein/terminator (A fluorescent protein cassette for monitoring transfection and transient expression and sorting and selecting transfected cells)
Cassette 7: other RNAi cassettes to interfere with RNA transportation, RNA degradation, et al. Other RNA interference methods like siRNA can also be used with the system.
Cassette 8: promoter/gene for ZFN or TALEN/terminator
Cassette 9: promoter/ssDNA binding proteins/terminator

The most basic components of the transient expression system of the invention are GSMS and reverse transcriptase expression cassettes, which can be included in the same or different expression vector, or it can be even included in the same expression cassette. Preferably, a natural or artificial tRNA expression cassette can be added in the same or different expression vector, which can produce primer tRNA for initiation of reverse transcription. A ssDNA binding protein expression cassette can also be added to the system, which produces ssDNA proteins that bind to and stabilize ssGSMS cDNA and facilitate nuclear transport of ssGSMS cDNA. Adding a specially designed siRNA that targets the RNA transcript of the target sequence but spare the ssGSMS cDNA can be beneficial. The degradation of target gene RNA may send a feedback signal to the nucleus to stimulate transcription of the target gene, maintaining the target gene in unwound or under-wound state. This provides more opportunity for ssGSMS cDNA to get access to the target gene in the chromosome. Additionally, expression cassette with sequence targeting endonuclease can be added to increase the efficiency of homology-based gene alteration. As shown in FIG. 12, one or more proteins along with GSMS can be combined and integrated in the same expression cassette.

The above mentioned expression cassettes can be constructed in the same vector, or they can be constructed in different vectors and co-introduced into the host cells. The vector of the transient expression system can be any vector suitable for RNA transcription and protein expression. It can be DNA or RNA, circular or linear, viral or non-viral vectors. The methods of introducing the expression vectors inside cells are known to people skilled in the art. Examples of such methods include electroporation, polyethylene glycol (PEG)-mediated transformation, DEAE-detran or CaPO₄ precipitation, liposome-based transfection, nanoparticle based transfection, viral infection, and direct injection. The hosts can be any suitable eukaryotic and prokaryotic organism, like mammals, plants, insects, algae, fungi, yeast, bacteria, cultured cells et al. The host genomic sequences subjected to modification can be any part of the genomic sequence including exons, introns, promoters, terminators, UTRs and regulatory sequences or motifs.

This technology may and can be used to treat infectious diseases, genetic disorders and other diseases like cancer; develop new traits in crops, forestry, livestock and fishery; create new cell lines in bacteria, yeast, algae, insects and mammals for drug and trait screening, fermentation, oil, protein, and all other medicinal, biological and industrial material production and scientific research.

### EXAMPLES

The following examples are provided by way of illustration only, not by way of limitation.

### Experiment 1

### Restoration of GFP Gene Using GSMS ssDNAs

In this example, a GFP GSMS ssDNA is used to correct a frame shift deletion in a GFP mutant gene stably integrated in the genome of yeast cells. A complete GFP coding sequence with only one frame shift deletion close to 5′ end is stably integrated into yeast cells using conventional cloning methods known to one skilled in the art (Molecular Cloning: a Laboratory Manual, 3rd Ed., 2001, Cold Spring Harbor Laboratory Press) (Ausubel, 2005. Current Protocols in Molecular Biology. Greene Publishing Associates and Wiley-Interscience). The GFP mutant sequence in the yeast genome is confirmed by DNA sequencing. The frame shift deletion disrupts the function of GFP and correction of the frame shift deletion will restore the function of GFP and make yeast cells fluorescent.

A GSMS expression cassette is constructed by linking a strong constitutive yeast promoter (e.g. TEF1 and PKG1 promoter) to a 400 bp wild type GFP coding sequence encompassing the deletion site of the mutant GFP, followed by a primer binding sequence and a yeast transcription terminator sequence. The 400 bp wild type GFP coding sequence lacks the start codon and is not able to be translated into protein by itself. Only correction of the GFP mutant in the nuclear DNA can lead to a functional fluorescent protein. The primer binding sequence of GSMS has 18 base pairs complementary to the 3′ end sequence of tRNA^{pro}, which is the tRNA primer used by M-MuLV reverse transcriptase. Since this example requires production of an exact piece of wild type GFP coding sequence to correct the error in mutant GFP sequence, a reverse transcriptase with high fidelity (e.g. SuperScript™II reverse transcriptase) is used to construct the reverse transcriptase expression cassette. The reverse transcriptase expression cassette contains a strong yeast promoter, a gene encoding SuperScript™ II M-MuLV reverse transcriptase, and a yeast transcription terminator sequence. A tRNA^{pro} expression cassette can be constructed by including a yeast promoter operatively linked to a DNA sequence encoding the tRNA^{pro}, followed by a transcription terminator sequence.

E. coli.

RecA protein and its functional and structural homolog in other species are key proteins for homologous recombination that mediate homology recognition and homologous DNA pairing and strand exchange. RecA and its eukaryotic homology RAD51 can bind to ssDNA to form a dynamic nucleoprotein filament, perform homology search along dsDNA strand, and facilitate homologous DNA strand invasion. A yeast RAD51 expression cassette can either be constructed as a separate expression cassette or it can be combined with GSMS expression cassette into a combination expression cassette as shown in FIG. 12. In the combination expression cassette, a yeast promoter is operatively linked to a RAD51 coding DNA sequence, followed by a hairpin forming sequence and a GSMS sequence, and a transcription terminator at the 3′ end. The combined RAD51 and GSMS expression cassette can result in coupled expression of RAD51 protein and ssGSMS cDNA in close proximity, which facilitates the formation of GSMS-RAD51 nucleoprotein filament, homology search of GFP sequence, and GSMS strand invasion and correction of GFP mutant gene.

The above expression cassettes can be included in one single or different yeast vectors. The yeast vectors can be circular or linear, but integrative vectors should be avoided so as to reduce the possibility of integration of vectors themselves into the yeast genome. The vector(s) containing SuperScript™II reverse transcriptase, RAD51, and GSMS expression cassettes can be introduced to yeast cells with the GFP mutant gene using methods well known to one skilled in the art (Ausubel, 2005), for example, LiAc/SS Carrier DNA/PEG method (Gietz, 2007), electroporation, and enzyme digestion. The correction of nuclear GFP mutant gene results in a functional fluorescent GFP protein that can be detected in situ using a confocal microscope. Or cells with corrected GFP gene can be collected using flow cytometry and the sequence of the corrected GFP gene can be directly determined by DNA sequencing.

### Experiment 2

### Knockout of GFP Gene Using GSMS ssDNAs and GFP siRNA

This example illustrates using GSMS ssDNA to knockout a functional GFP gene in a mammalian cell. A mammalian cell line (e.g. HEK 293T cells) is stably transfected and selected for green fluorescent cells with nuclear integration of a normal and functional GFP cDNA (293T-GFP cells). The functional GFP protein can be disrupted by introduction of a frame shift deletion and a pre-mature stop codon into the GFP gene via homologous recombination. A GSMS is designed to contain a frame shift insertion and two pre-mature stop codons inserted into a 300 bp GFP coding sequence close to the 5′ end of GFP cDNA. The primer binding sequence of GSMS (+) strand contains a stretch of 20 dT residues at the 3′ end, which will be transcribed into a stretch of 20 uracil residues at 3′ end of the GSMS RNA. At the end of transcription, a poly(A) tail is added to the 3′ end of GSMS RNA transcripts, which can be self-annealed to the stretch of 20 uracil residues, acting as a primer to initiate reverse transcription. A double-stranded GFP siRNA is designed outside the 300 bp homologous region of GSMS, and preferably located close to 5′ of GFP cDNA. The GFP siRNA induces degradation of GFP mRNA, which may send a signal to keep the transcription of GFP gene active and maintain the genomic locus of GFP gene in unwound or under-wound state. A SuperScript™II reverse transcriptase expression cassette is constructed as in Example 1.

Chemically synthesized GFP siRNA and vectors containing GSMS and SuperScript™II reverse transcriptase expression cassette can be introduced into 293T-GFP cells using suitable transfection methods known to one skilled of the art, for example, using lipofectamine, calcium phosphate, DEAE-dextran based methods, gene gun, and electroporation. Culture the 293T-GFP cells under standard cell culture conditions for a few days and look for loss of green fluorescence as an indication of successful disruption of GFP proteins. The non-fluorescent cells can be separated from fluorescent cells using flow cytometry and PCR and DNA sequencing of the GFP gene in non-fluorescent cells can further verify whether successful knockout of GFP gene has occurred.

### Experiment 3

### Target a GFP Gene into a Recombination Hotspot Using GSMS ssDNA

This example illustrates using GSMS ssDNA to introduce a foreign gene into a specific locus in a host genome, more particularly to a recombination hotspot. First, identify the genomic sequence of the recombination hotspot to be targeted. The GSMS is designed to contain a functional GFP gene including a promoter region and a full length protein coding sequence flanked by 100 bp sequences complementary to the genomic sequence of the recombination hotspot. As described in Example 1, the primer binding sequence of GSMS has 18 base pairs complementary to the 3′ end sequence of tRNA^{pro}. A SuperScript™ II reverse transcriptase expression cassette and a combination expression cassette containing mammalian GAD51 protein coding sequence and GSMS is constructed as described in Example 1. Transfer vectors containing the above expression cassettes into a mammalian cell line and culture cells for at least one week until the transient expression vectors are degraded and disappear. Search for clones of green fluorescent cells and further confirm homologous integration vs. non-homologous integration using DNA sequencing.

### Experiment 4

### Build a Library of Cells with Random Mutations in a Target Genomic Sequence Using GSMS ssDNA and TALEN

This example illustrates a method of building a library of cells with random mutations in a target genomic locus using a GSMS ssDNA, reverse transcriptase with low proofreading ability, and a sequence targeting endonuclease (e.g. TALEN). This method can be applied to integrate random mutations into a DNA sequence of a targeted genomic locus and build a library of mutated cells for screening of new traits or desired phenotypes. The targeted genomic locus to be changed can be a continuous sequence of an exon region of a gene, a regulatory region, or a cDNA sequence integrated in the genome. Ideally, there is a trait or phenotype that can be easily screened for once the mutations are created inside cells. The GSMS is designed to contain a sequence identical to the target genomic sequence to be changed. The error prone HIV reverse transcriptase (average error rate: 1 in 1500 bases) is selected to generate random mutations in ssGSMS cDNA during reverse transcription. To match for HIV reverse transcriptase cognate tRNA₃^{Lys}, the primer binding sequence of GSMS contains a 18 bp sequence complementary to the 3′ end sequence of tRNA₃^{Lys}. The DNA binding domain of TALEN is customized to specifically recognize a region inside the target genomic sequence and will be used to create a double strand break within the target genomic sequence. Using TALEN to create a double strand break inside host cells can greatly increase the efficiency of homologous integration, enabling creation of a population of cells each with a different mutated sequence. GSMS, HIV reverse transcriptase, GAD51, and TALEN expression cassettes are created as described in Example 1. Vectors containing GSMS, HIV reverse transcriptase, and TALEN expression cassettes are introduced into host cells using methods well known to one skilled in the art. Once inside cells, the GSMS expression cassette produces a GSMS RNA, which is reverse transcribed into ssGSMS cDNA with random mutations. ssGSMS cDNA binds to GAD51 protein to form a nucleoprotein filament and is transported to the nucleus. Expressed TALEN enzyme enters the nucleus and creates a double strand break within the target genomic sequence. GSMS-GAD51 nucleoprotein filament performs a homology search, anneals to the homologous region of the target genomic sequence, and results in homologous integration of GSMS with random mutations.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

### REFERENCES

A H Lund, M. D. (1997, February). Complementation of a Primer Binding Site-Impaired Murine Leukemia Virus-Derived Retroviral Vector by a Genetically Engineered tRNA-Like Primer. JOURNAL OF VIROLOGY, 1191-1195.
Andersen M S, Sorensen C B, Bolund L, Jensen T G (2002) Mechanisms underlying targeted gene correction using chimeric RNA/DNA and single-stranded DNA oligonucleotides. J. Mol. Med. 80: 770-781.
Andrew J. Wood, T.-W. L. (2011). Targeted Genome Editing Across Species Using ZFNs and. Science (333), 6040.
Arnould, Sylvain, et al. (2006). “Engineering of Large Numbers of Highly Specific Homing Endonucleases that Induce Recombination on Novel DNA Targets”. Journal of Molecular Biology 355 (3): 443-58.
Ausubel, F. M., Brent, R., Kingston, R. E., Moore, D. D., Seidman, J. G., Smith, J. A., and Struhl, K. (2005). Current Protocols in Molecular Biology (New York: Greene Publishing Associates and Wiley-Interscience).
Bae K H, Kwon Y D, Shin H C, Hwang M S, Ryu E H, Park K S, Yang H Y, Lee D K, Lee Y, Park J et al. (2003) Human zinc fingers as building blocks in the construction of artificial transcription factors. Nat Biotechnol 21(3): 275-280.
Battula N, L. L. (1976). On the fidelity of DNA replication, lack of endodeoxyribonucleases activity and error-correcting function in avian myeloblastosis virus DNA polyerase, J. Biol Chem (251), 982-6.
Battula N, L. L. (1974). The infidelity of avian myeloblastosis virus deoxyribonucleic acid polymerase in polynucleotide replication. J. Biol Chem (249), 4086-93.
Bennetzen, A. K. (1999). Plant Retrotransposons. Annu. Rev. Genet, 33, 479-532.
Blackburn, G. M. (2006). Covalent Interactions of Nucleic Acids with Small Molecules and Their Repair. In G. M. Blackburn, Nucleic acids in chemistry and biology (pp. 295-339). Cambridge: RSC Pub.
Blackurn, G. M. (2006). RNA Structure and Function. In G. M. Blackurn, Nucleic acids in chemistry and biology (pp. 253-250). Cambridge: RSC Pub.
Boeke, J. D. (1989). Transcription and reverse transcription of retrotransposons. Annu. Rev. Microbiol., 43, 403-34.
Brummelkamp T R, Bernards R, Agami R. (2002) A system for stable expression of short interfering RNAs in mammalian cells. Science 296: 550-3.
Brown, J. W. (1996). Arabidopsis intron mutations and pre-mRNA splicing. The Plant Journal, 10 (5), 771-780.
Campbell C R, Keown W, Lowe L, Kirschling D, Kucherlapati R (1989) Homologous recombination involving small single-stranded oligonucleotides in human cells. New Biol. 1: 223-227.
Chick H E, Nowrouzi A, Fronza R, McDonald R A, Kane N M, Alba R, Delles C, Sessa W C, Schmidt M, Thrasher A J, Baker A H. (2012) Integrase-deficient lentiviral vectors mediate efficient gene transfer to human vascular smooth muscle cells with minimal genotoxic risk. Hum Gene 23(12):1247-57.
Chuanxian Wei, Jiyong Liu, Zhongsheng Yu, Bo Zhang, Guanjun Gao, Renjie Jiao. (2013) TALEN or Cas9-Rapid, Efficient and Specific Choices for Genome Modifications. Journal of Genetics and Genomics, 40(6): 281-289
De Geest, B., Van Linthout, S., Lox, M., Collen, D. and Holvoet, P., (2000). Sustained expression of human apolipoprotein A-I after adenoviral gene transfer in C57B L/6 mice: Role of apolipoprotein A-I promoter, apolipoprotein A-I introns, and human apolipoprotein E enhancer. Hum. Gene Ther., 11: 101-112.
de Semir D, Aran J M (2006) Targeted gene repair: the ups and downs of a promising gene therapy approach. Curr. Gene Ther. 6: 481-504.
DUESBERG, D. W. (1990). Retroviral recombination during reverse transcription. Proc. Natl. Acad. Sci. USA, 87, 2052-2056.
Engstrom J U, Kmiec E B (2008) DNA replication, cell cycle progression and the targeted gene repair reaction. Cell Cycle 15: 1402-1414.
Fisher T L, Terhorst T, Cao X, Wagner R W. (1993) Intracellular disposition and metabolism of fluorescently-labeled unmodified and modified oligonucleotides microinjected into mammalian cells. Nucleic Acids Res. 21(16):3857-65.
Gietz R D, Schiestl R H. (2007) High-efficiency yeast transformation using the LiAc/SS carrier DNA/PEG method. Nat Protoc. 2(1):31-4.
Halpin C, Cooke S E, Barakate A, El Amrani A, Ryan M D (1999) Self-processing 2A-polyproteins: a system for co-ordinate expression of multiple proteins in transgenic plants. Plant J 17: 453-459.
Hauser, M. A., Robinson, A., Hartigan-O'Connor, D., Williams-Gregory, D., Buskin, J. N., Apone, S., Kirk, C. J., Hardy, S., Hauschka, S. D. and Chamberlain, J. S., (2000). Analysis of muscle creatine kinase regulatory elements in recombinant adenoviral vectors. Mol. Ther., 2: 16-25.
Holthausen J T, Wyman C, Kanaar R. (2010) Regulation of DNA strand exchange in homologous recombination. DNA Repair. 9(12):1264-72.
Hong-Xiang Liu, W. F. (1996). Mapping of branchpoint nucleotides in mutant pre-mRNAs expressed in plant cells. The Plant Journal, 9 (3), 381-389.
Igoucheva O, Alexeev V, Yoon K (2004a) Oligonucleotide directed mutagenesis and targeted gene correction: a mechanistic point of view. Curr. Mol. Med. 4: 445-463.
J. W. S. Brown, C. G.-E. (2002). Splicing signals and factors in plant intron removal. Biochemical Society Transactions, 30 (Part 2), pp. 146-149.
Leonetti J P, Mechti N, Degols G, Gagnor C, Lebleu B. (1991) Intracellular distribution of microinjected antisense oligonucleotides. Proc Natl Acad Sci USA. 88(7):2702-6.
KLEIMAN, J. M. (1997). Primer tRNAs for Reverse Transcription. JOURNAL OF VIROLOGY, 71 (11), 8087-8095.
Kim E, Kim S, Kim D H, Choi B S, Choi I Y, Kim J S. (2012) Precision Genome engineering with programmable DNA-nicking enzymes. Genome Res. 22(7):1327-33.
Kim, I.-H., Jozkowicz, A., Piedra, P. A., Oka, K. and Chan, L., (2001). Lifetime correction of genetic deficiency in mice with a single injection of helper-dependent adenoviral vector. PNAS, 98: 13282-13287.
Kim J H, Lee S R, Li L H, Park H J, Park J H, Lee K Y, Kim M K, Shin B A, Choi S Y. (2011) High cleavage efficiency of a 2A peptide derived from porcine teschovirus-1 in human cell lines, zebrafish and mice. PLoS One. 6(4):e18556.
Kunkel T A, E. F. (1981). Deoxynucleoside [1-thio]triphosphates prevent proof-reading during in vitro DNA synthesis. Proc Natl Acad Sci USA, 78, 6734-8.
Li X, Heyer W D. (2008) Homologous recombination in DNA repair and DNA damage tolerance. Cell Res. 18(1):99-113.
Long, M. D. (1999). Intron-exon structures of eukaryotic model organisms. Nucleic Acids Research, 27 (15), 3219-3228.
Neville E. Sanjana, L. C. (2013). A Transcription Activator-Like Effector (TALE) Toolbox for. Nat Protoc., 7 (1), 171-192.
Parekh-Olmedo H, Kmiec E B (2007) Progress and prospects: targeted gene alteration (TGA). Gene Ther. 14: 1675-1680.
R Marquet, C. l. (1995). tRNAs as primer of reverse transcriptases. Biochimie, 77, 113-124. Robyn L. Maher, S. W. (2013). Coordinated Binding of Single-Stranded and Double-. PLoS ONE, 8 (6), e6654.
S. B. Sandmeyer, T. M. (1996). Morphogenesis at the Retrotransposon-Retrovirus Interface: Gypsy and Copia Families in Yeast and Drosophila. In H.-G. Krä usslich, Morphogenesis and Maturation of Retroviruses (Vol. 214, pp. 261-296). Springer Berlin Heidelberg.
Saïb, S, N. (2004). Early steps of retrovirus replicative cycle. Retrovirology, 1 (9).
Sainis, R. K. (2010). Plant DNA Recombinases: A LongWay to Go. Journal of Nucleic Acids.
Skarli, M., Kiri, A., Vrbova, G., Lee, C. A. and Goldspink, G., (1998). Myosin regulatory elements as vectors for gene transfer by intramuscular injection. Gene Ther., 5: 514-520.
Smith, J.; Grizot, S.; Arnould, S.; Duclert, A.; Epinat, J.-C.; Chames, P.; Prieto, J.; Redondo, P. et al. (2006). “A combinatorial approach to create artificial homing endonucleases cleaving chosen sequences”. Nucleic Acids Research 34 (22): e149.
Sui G, Soohoo C, Affar E B, Gay F, Shi Y, Forrester W C, Shi Y. (2002) A DNA vector-based RNAi technology to suppress gene expression in mammalian cells. Proc Natl Acad Sci USA 99: 5515-20.
Voytas, D. (1993). Yeast retrotranscopsons and tRNAs. Trends in Genetics, 9, 421-426.
Wakefield, J. (1995). Human immunodeficiency virus type 1 can use different tRNAs as primers for reverse transcription but selectively maintains a primer binding site complementary to tRNA (3Lys). J. Virol, 69, 6021-6029.
Zhang Y, Zhang F, Li X, Baller J A, Qi Y, Starker C G, Bogdanove A J, Voytas D F. (2013). Transcription Activator-Like Effector Nucleases Enable efficient plant genome engineering. Plant Physiology, 161, 20-17.
Zaki, E. A. (2003). Plant retroviruses: structure, evolution and future applications. African Journal of Biotechnology, 2 (6), 136-139.
Zhang Y, Zhang F, Li X, Baller J A, Qi Y, Starker C G, Bogdanove A J, Voytas D F. (2013) Transcription Activator-Like Effector Nucleases Enable Efficient Plant Genome Engineering. Plant Physiol. 161:20-27.
Zorin B, Hegemann P, Sizova I. (2005) Nuclear-gene targeting by using single-stranded DNA avoids illegitimate DNA integration in Chlamydomonas reinhardtii. Eukaryot Cell. 4(7): 1264-72.
Zhu T, Mettenburg K, Peterson D J, Tagliani L, Baszczynski C L. Engineering herbicide-resistant maize using chimeric RNA/DNA oligonucleotides. Nat Biotechnol. 2000 May; 18(5):555-8.

What is claimed is:

1. A system for introducing an alteration in a target genomic sequence of a host cell, comprising:
a) a Genomic Sequence Modification Sequence (GSMS) expression cassette, wherein said GSMS expression cassette comprises a polynucleotide sequence homologous to said target genomic sequence and a primer binding sequence, and wherein said GSMS expression cassette produces GSMS RNAs;
b) a reverse transcriptase expression cassette, wherein said reverse transcriptase cassette comprises a polynucleotide sequence encoding a reverse transcriptase gene; and
c) a means of co-introducing said GSMS expression cassette and said reverse transcriptase expression cassette into said host cells, whereby said GSMS RNAs are reverse transcribed to single stranded GSMS cDNAs (ssGSMS cDNA) by said reverse transcriptase, and said ssGSMS cDNAs direct said alteration in said target genomic sequence.

2. The system of claim 1, further comprising a means of selecting host cells with altered target genomic sequence.

3. The system of claim 1, wherein said ssGSMS cDNA directs alteration in said target genomic sequence via DNA repair pathway or homologous recombination.

4. The system of claim 1, wherein said GSMS comprises a sequence fully complementary to said target genomic sequence.

5. The system of claim 1, wherein said GSMS comprises a sequence fully complementary to said target genomic sequence, except for one or more nucleotide differences at preselected positions of said GSMS, wherein said nucleotide difference is a mismatch, a deletion, an insertion, or a combination of the above.

6. The system of claim 1, wherein said GSMS comprises a heterologous polynucleotide sequence flanked by sequences homologous to said host genomic sequence.

7. The system of claim 6, wherein said heterologous polynucleotide sequence encoding a selectable marker.

8. The system of claim 1, wherein said GSMS comprises a sequence homologous to a genomic sequence within a recombination hotspot region.

9. The system of claim 1, wherein said GSMS comprises a sequence homologous to said target genomic sequence having direct or inverted repeats.

10. The system of claim 1, wherein said primer binding sequence of said GSMS is complementary to 3′ end of a natural tRNA or an artificial tRNA sequence.

11. The system of claim 1, wherein 5′ end of said GSMS RNA can form a secondary structure that terminates the reverse transcription when said reverse transcriptase meets said secondary structure.

12. The system of claim 1, wherein said reverse transcriptase is a naturally occurred reverse transcriptase or an engineered reverse transcriptase.

13. The system of claim 1, wherein said reverse transcriptase has good proof-reading ability.

14. The system of claim 1, wherein said reverse transcriptase has poor proof-reading ability.

15. The system of claim 1, further comprising a primer expression cassette, wherein said primer expression cassette produces a natural or artificial primer tRNA that can bind to said primer binding sequence of said GSMS RNAs to initiate the reverse transcription, and a means of co-introducing said GSMS, said reverse transcriptase, and said primer expression cassette into said host cells.

16. The system of claim 1, further comprising
a) a single stranded DNA (ssDNA) binding protein expression cassette, wherein said ssDNA binding protein expression cassette encodes a ssDNA binding protein; and
b) a means of co-introducing said GSMS, said reverse transcriptase, and said ssDNA binding protein expression cassettes into said host cells.

17. The system of claim 16, wherein said ssDNA binding protein is selected from the group consisting of replication protein A, RecA, Rad51, DMC1, ICP8, SSB, and proteins homologous to said replication protein A, said RecA, said Rad51, said DMC1, said ICP8 and said SSB.

18. The system of claim 1, further comprising
a) a sequence targeting endonuclease expression cassette, wherein said sequence targeting endonuclease expression cassette encodes a sequence targeting endonuclease, and wherein said sequence targeting endonuclease comprises a sequence recognition domain and a DNA cleavage nuclease domain, and wherein said sequence targeting endonuclease targets the same homologous region of said target genomic sequence as said GSMS; and
b) a means of co-introducing said GSMS, said reverse transcriptase, and said sequence targeting endonuclease expression cassettes into said host cells.

19. The system of claim 18, wherein said sequence recognition domain of said sequence targeting endonuclease is selected from the group consisting of Zinc Finger DNA binding motifs, Transcription Activator-like effectors DNA binding domains, and meganuclease sequence recognition domains.

20. The system of claim 18, wherein said DNA cleavage nuclease domain of said sequence targeting endonuclease cuts a double-stranded polynucleotide sequence and creates a double strand break.

21. The system of claim 18, wherein said DNA cleavage nuclease domain of said sequence targeting endonuclease nicks at a double-stranded polynucleotide sequence and cuts only one strand of said double-stranded polynucleotide sequence.

22. The system of claim 1, further comprising
a) a siRNA expression cassette, wherein said siRNA expression cassette produces a siRNA that induces the degradation of the mRNA of said target genomic sequence while siRNA does not have sequence homology with said GSMS RNAs; and
b) a means of co-introducing said GSMS, said reverse transcriptase, and said siRNA expression cassette into said host cells, whereby said siRNA keeps said target genomic sequence in a unwound state and increases chances of interaction of said ssGSMS cDNA to said target genomic sequence.

23. The system of claim 22, wherein said siRNA is chemically synthesized and is co-introduced into said host cells along with said GSMS and said reverse transcriptase expression cassette.

24. The system of claim 1, further comprising one or more expression cassettes selected from the group consisting of a primer expression cassette, a ssDNA binding protein expression cassette, a sequence targeting endonuclease expression cassette, and a siRNA expression cassette.

25. The system of claim 1, further comprising a combination expression cassette, wherein a single promoter is operatively linked to two or more protein coding sequences, and wherein adjacent protein coding sequences are separated by a translational skipping sequence.

26. The system of claim 1, further comprising a combination expression cassette, wherein a single promoter is operatively linked to two or more protein coding sequences and is further linked to GSMS, wherein adjacent protein coding sequences are separated by a translational skipping sequence, and GSMS and its upstream protein coding sequences are separated by a sequence encoding a RNA with a hairpin structure.

27. The system of claim 1, wherein said GSMS and reverse transcription expression cassette is a DNA or a RNA.

28. A method of introducing an alteration in a target genomic sequence of a host cell, comprising the steps of:
a) constructing a GSMS expression cassette, wherein said GSMS comprises a polynucleotide sequence homologous to said target genomic sequence and a primer binding sequence, and wherein said GSMS expression cassette produces GSMS RNAs;
b) constructing a reverse transcriptase expression cassette, wherein said reverse transcriptase cassette encodes a reverse transcriptase; and
c) co-introducing said GSMS expression cassette and said reverse transcriptase expression cassette into said host cells, whereby said GSMS RNAs are reverse transcribed to ssGSMS cDNAs by said reverse transcriptase, and said ssGSMS cDNAs direct said alteration in said target genomic sequence.

29. The method of claim 28, further comprising selecting host cells with altered target genomic sequence.

30. The method of claim 28, further comprising co-introducing into said host cells one or more expression cassettes selected from the group consisting of a primer expression cassette, a ssDNA binding protein expression cassette, a sequence targeting endonuclease expression cassette, and a siRNA expression cassette.

31. A method of obtaining a population of cells with random mutations in a target genomic sequence, comprising the steps of:
a) constructing a GSMS expression cassette, wherein said GSMS comprises a polynucleotide sequence fully complementary to said target genomic sequence and a primer binding sequence, and wherein said GSMS expression cassette produces GSMS RNAs;
b) constructing a reverse transcriptase expression cassette, wherein said reverse transcriptase cassette produces s a reverse transcriptase with poor proofreading ability;
c) co-introducing said GSMS expression cassette and said reverse transcriptase expression cassette into said host cells, whereby said GSMS RNAs are reverse transcribed to ssGSMS cDNAs with random mutations by said reverse transcriptase with poor proofreading ability, and said ssGSMS cDNAs direct the integration of random mutations into said target genomic sequence; and
d) collecting cells with random mutations in said target genomic sequence.

32. The method of claim 31 further comprising co-introducing into said host cells one or more expression cassettes selected from the group consisting of a primer expression cassette, a ssDNA binding protein expression cassette, a sequence targeting endonuclease expression cassette, and a siRNA expression cassette.
